(19) United States
(12) Patent Application Publication
(10) Pub. No.: US 2023/0365505 A1
(43) Pub. Date: Nov. 16, 2023

(54) PROCESSES FOR THE PREPARATION OF IVABRADINE HCl POLYMORPHS

(71) Applicant: CAMBREX PROFARMACO MILANO s.r.l., Paullo (MI) (IT)

(72) Inventors: Cristina CIANCIMINO, Paullo (MI) (IT); Piero DANELLI, Paullo (MI) (IT); Elios GIANNINI, Paullo (MI) (IT); Craig CALLAHAN, Edinburgh Technopole (GB); Daniele VIGO, Paullo (MI) (IT); Oreste PICCOLO, Sirtori (LC) (IT)

(21) Appl. No.: 18/029,038

(22) PCT Filed: Oct. 25, 2021
(86) PCT No.: PCT/EP2021/079510

§ 371 Date: Mar. 28, 2023

(30) Foreign Application Priority Data
Oct. 26, 2020 (IT) 102020000025312

(51) Int. Cl. C07D 223/16

(52) U.S. Cl. CPC C07D 223/16; C07B 2200/13

(57) ABSTRACT

Disclosed are new processes for the preparation of known polymorphs of ivabradine HCl, such processes being characterized by robust protocols suitable for industrial production.

### CROSS-REFERENCE TO RELATED APPLICATIONS

This application is the U.S. national phase of International Application No. PCT/EP2021/079510 filed Oct. 25, 2021, which designated the U.S. and claims priority to IT 102020000025312 filed Oct. 26, 2020, the entire contents of each of which are hereby incorporated by reference.

### TECHNICAL FIELD

The present invention relates to new processes for the preparation of known polymorphs of ivabradine HCl said processes being characterized by robust protocols suitable for industrial production.

### BACKGROUND ART

Ivabradine HCl (S)-3-(3-(((3,4-dimethoxybicyclo[4.2.0]octa-1,3,5-trien-7-yl)methyl)(methyl)amino)propyl)-7,8-dimethoxy-1,3,4,5 -tetrahydro-2H-benzo[d]azepin-2-one hydrochloride of formula I is a useful drug for the treatment of cardiovascular diseases, such as angina pectoris, myocardial infarction and associated rhythm diseases. Ivabradine HCl has bradycardic properties, which make it particularly useful in the treatment or prevention of supraventricular rhythm disorders and heart failure.

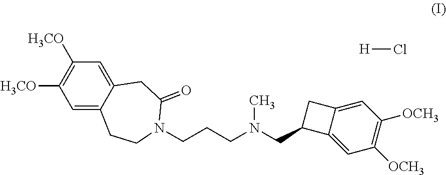 [SMILES: COc1cc2c(cc1OC)CC(=O)N(CCCN(C)C[C@H]1Cc3cc(OC)c(OC)cc31)CC2 ; Cl]

Ivabradine HCl was initially obtained in crystalline form (U.S. Pat. No. 5,296,482; EP 0534859) by treating the corresponding free base with 0.1 N HCl and recrystallization after evaporation of the mixture from acetonitrile with a yield of 55%.

Ivabradine HCl exists in several polymorphic forms characterized by specific

XRPDs: in particular forms I, II, IV, X, Z, K, C, S, alpha, beta, gamma, delta, gamma-d, beta-d and delta-d, are described respectively in U.S. Pat. No. 8,541,405, CN 103 183 639, U.S. Pat. No. 9,139,531, WO 2011/098582, U.S. Pat. No. 9,120,755, CN 103 012 269, CN 103 864 690, U.S. Pat. No. 7,176,197, U.S. Pat. No. 7,361,649, U.S. Pat. No. 7,361,650, U.S. Pat. No. 7,358,240, U.S. Pat. No. 7,361,651, U.S. Pat. No. 7,361,652 and U.S. Pat. No. 7,384,932.

The delta crystalline form of ivabradine HCl described in U.S. Pat. No. 7,358,240 is prepared by crystallizing the product obtained according to U.S. Pat. No. 5,296,482 from acetonitrile and isolating the crystalline form from the reaction mixture, after waiting for 2 days, by filtration and drying at room temperature and humidity. According to the analysis, the obtained delta crystalline form is a hydrate characterized by a water content of about 2.8% and an acetonitrile content between 1% and 5%, more often between 1.5% and 3%.

The solids prepared and isolated, obtained according to the cited prior art, after the crystallization of ivabradine HCl from acetonitrile, comprise adsorbed or solvated acetonitrile.

Acetonitrile is a class 2 solvent and its content in pharmaceutical products has a limit of 410 ppm as reported in the ICH guidelines and must therefore be adequately removed from any product intended to be formulated in pharmaceutical compositions.

The delta form obtained from acetonitrile is however a very stable acetonitrile solvate and even if subjected to drying under very high temperature, vacuum and time conditions, it is not possible to observe a complete transition to the anhydrous delta-d form.

The delta-d form of ivabradine HCl is the polymorph described in U.S. Pat. No. 7,384,932, characterized by XRPD having the characteristic peaks reported in the following table:

| | | | Area | | Inter- |
|---|---|---|---|---|---|
| | Angle | | (counts | | planar |
| Line | 2 theta | Height | × | FWHM | distance |
| no. | (degrees) | (counts) | degrees) | (degrees) | (Å) |
| 1 | 4.1 | 414 | 41 | 0.1004 | 21.672 |
| 2 | 6.8 | 176 | 139 | 0.8029 | 13.078 |
| 3 | 8.6 | 1020 | 101 | 0.1004 | 10.305 |
| 4 | 9.1 | 323 | 43 | 0.1338 | 9.687 |
| 5 | 10.9 | 224 | 30 | 0.1338 | 8.100 |
| 6 | 11.7 | 354 | 47 | 0.1338 | 7.592 |
| 7 | 14.6 | 2774 | 458 | 0.1673 | 6.074 |
| 8 | 15.3 | 1805 | 328 | 0.184 | 5.800 |
| 9 | 16.6 | 986 | 163 | 0.1673 | 5.345 |
| 10 | 17.2 | 3821 | 946 | 0.2509 | 5.153 |
| 11 | 18.1 | 2290 | 378 | 0.1673 | 4.898 |
| 12 | 19.1 | 440 | 73 | 0.1673 | 4.649 |
| 13 | 19.6 | 289 | 38 | 0.1338 | 4.526 |
| 14 | 20.1 | 650 | 86 | 0.1338 | 4.408 |
| 15 | 20.9 | 887 | 146 | 0.1673 | 4.252 |
| 16 | 21.4 | 3112 | 565 | 0.184 | 4.147 |
| 17 | 22.1 | 1708 | 254 | 0.1506 | 4.027 |
| 18 | 22.5 | 1191 | 275 | 0.2342 | 3.945 |
| 19 | 23.4 | 619 | 102 | 0.1673 | 3.800 |
| 20 | 23.9 | 1343 | 222 | 0.1673 | 3.728 |
| 21 | 24.7 | 256 | 34 | 0.1338 | 3.604 |
| 22 | 25.6 | 309 | 41 | 0.1338 | 3.482 |
| 23 | 26.2 | 1899 | 313 | 0.1673 | 3.397 |
| 24 | 26.9 | 1588 | 183 | 0.1171 | 3.310 |
| 25 | 27.6 | 1357 | 224 | 0.1673 | 3.231 |
| 26 | 29.1 | 140 | 37 | 0.2676 | 3.069 |
| 27 | 29.5 | 145 | 29 | 0.2007 | 3.023 |

The process for the preparation of the delta-d form of ivabradine HCl, described in U.S. Pat. No. 7,384,932, comprises a crystallization from acetonitrile followed by drying at 85° C. Said process is not very effective in terms of purity and stability of the final product which has a rather high residual acetonitrile content.

Other processes are known for the preparation of crystalline forms of ivabradine HCl and in particular of the delta form and of the anhydrous delta-d form.

U.S. Pat. No. 9,440,924 describes a process for the preparation of crystalline forms of ivabradine HCl which comprises the formation of a crystalline acetone solvate of ivabradine HCl starting from another solvate, followed by the treatment of the above acetone solvate in an atmosphere with relative humidity around 50%, for the preparation of the delta form or, for subsequent drying, the anhydrous form delta-d.

U.S. Pat. No. 9,440,924 therefore teaches the need to pass through the formation of acetone solvate, even starting from other solvates with solvents other than acetone, to obtain the desired crystalline form and in particular for the preparation of delta and delta-d forms.

In particular, it teaches that passing through other solvates does not lead to the anhydrous delta-d form of ivabradine HCl and that its preparation is bound to the formation of the acetone solvate.

In the examples reported in the above mentioned document, ivabradine HCl (delta-d form) is suspended in acetone, then cooled in a refrigerator overnight and filtered to give the corresponding acetone solvate. Said acetone solvate is dried under vacuum at 70° C. for 14 hours to yield delta-d crystalline form of ivabradine HCl.

CN 105 503 726 describes a process for the preparation of the anhydrous delta-d form of ivabradine HCl characterized by the dissolution of ivabradine HCl in a solvent selected from acetone, methyl ethyl ketone and methyl isobutyl ketone, followed by heating at 30-45° C. for 6-50 hours and subsequent filtration and drying in an inert atmosphere at 40-85° C.

CN'726 teaches that the control of the water content in the reagents and solvents of the preparation process of the delta-d form of ivabradine HCl, starting from polymorphic forms alpha, delta, II, III and IV, is advantageous in economic terms and stability of the delta-d form thus obtained. It is evident from the examples relating to the invention and from the reported comparative examples that to obtain the desired delta-d form it is necessary to have a very precise control of the reaction conditions, in particular of the water content of the starting materials, of the solvents, as well as of the temperatures and reaction times, in order to avoid the formation of other polymorphic forms.

It is also known that the use of alcoholic solvents in the crystallization phase of ivabradine HCl leads to the formation of polymorphs other than the delta and delta-d forms.

In particular U.S. Pat. No. 7,872,001 describes the preparation of the gamma-d form of ivabradine HCl by crystallization from a mixture of ethanol and water followed by drying to give the anhydrous form.

IN2016 2103 3046 and CN 103 012 269 describe the formation of ivabradine HCl in the alpha and C forms by treatment with ethanol mixed with esters.

U.S. Pat. No. 9,120,755 reports the preparation of polymorphic forms II and III for treatment of ivabradine HCl in the presence of solvents such as ethanol, isopropanol, methyl ethyl ketone, methyl isobutyl ketone or acetonitrile. Forms II and III obtained according to the described process do not contain other polymorphic forms such as beta, delta or gamma forms.

It is therefore clear that, for the preparation of delta-d forms of ivabradine HCl, the use of C1-C5 alcohols, such as ethanol, isopropanol, 2-methyl-2-butanol or methylethylketone, would have been against all expectation for the person skilled in the art.

The need therefore remains to find new processes that allow effective removal of the crystallization solvents in the preparation of the crystalline forms of ivabradine HCl, in particular for the preparation of the anhydrous crystalline form delta-d.

### BRIEF DESCRIPTION OF THE FIGURES

FIG. 1a: XRPD delta-d form of ivabradine HCl

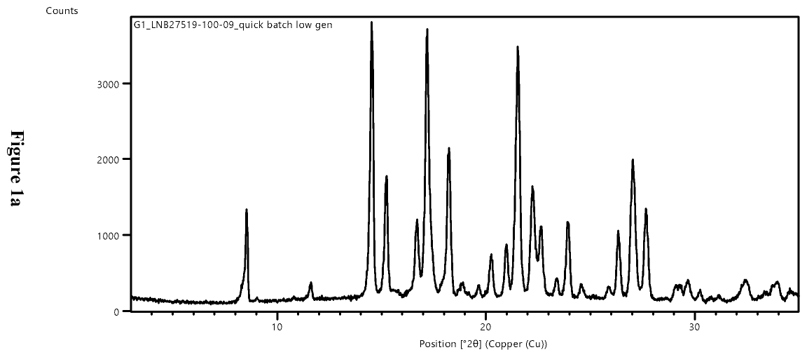

FIG. 1b: Table with list of peaks related to XRPD of FIG. 1a

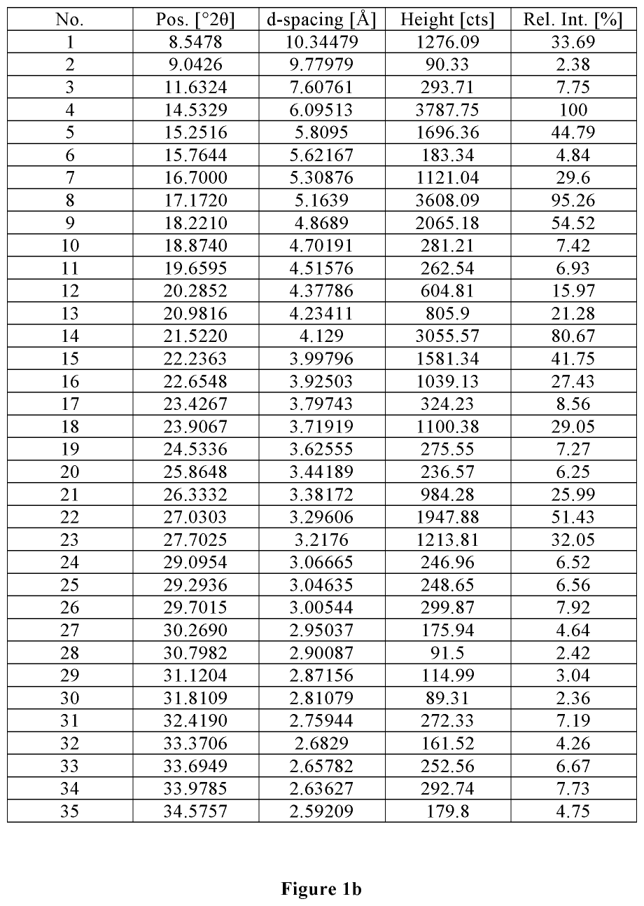

FIG. 2: XRPD delta-d form of ivabradine HCl with peaks indication

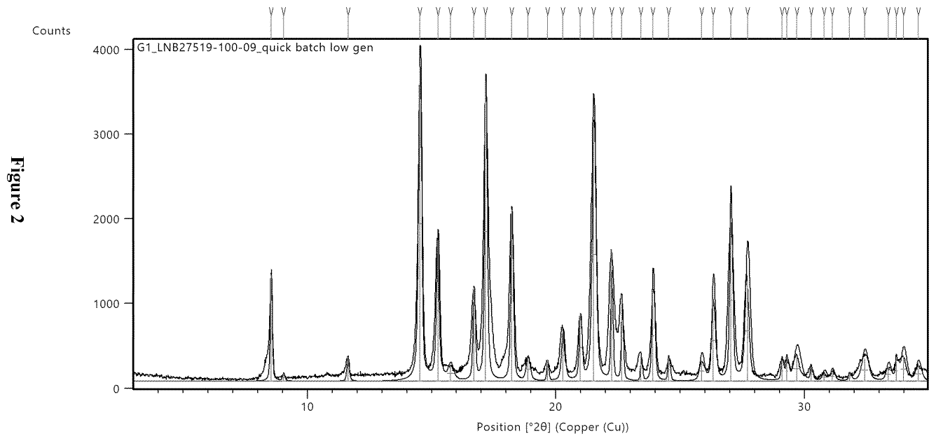

FIG. 3a: XRPD delta-hydrated form of ivabradine HCl

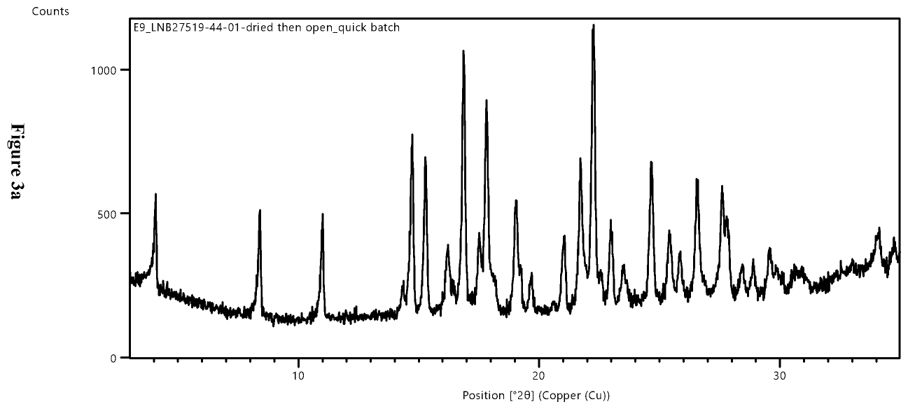

FIG. 3b: Table with list of peaks related to XRPD of FIG. 3a

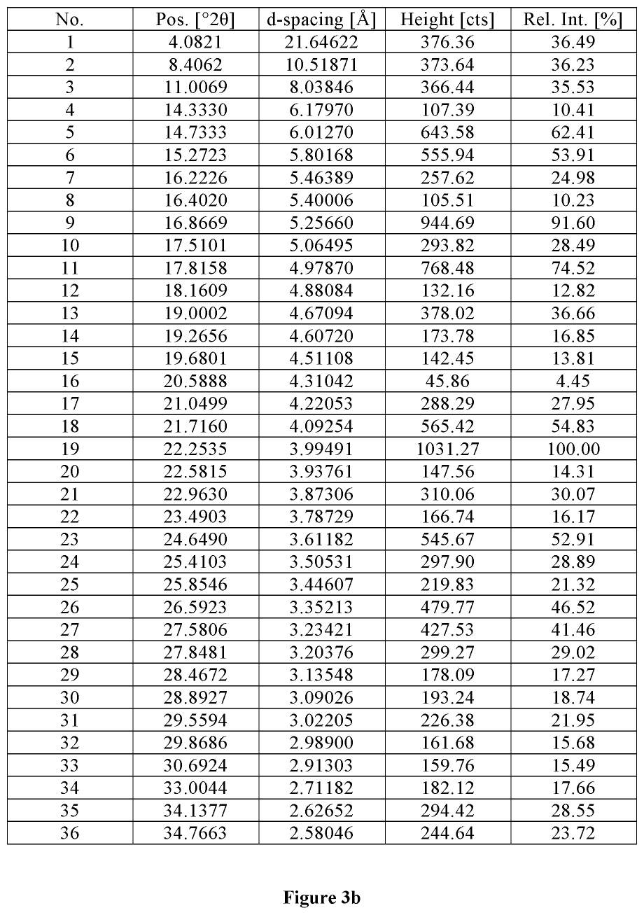

FIG. 4: XRPD delta-hydrated form of ivabradine HCl with peaks indication

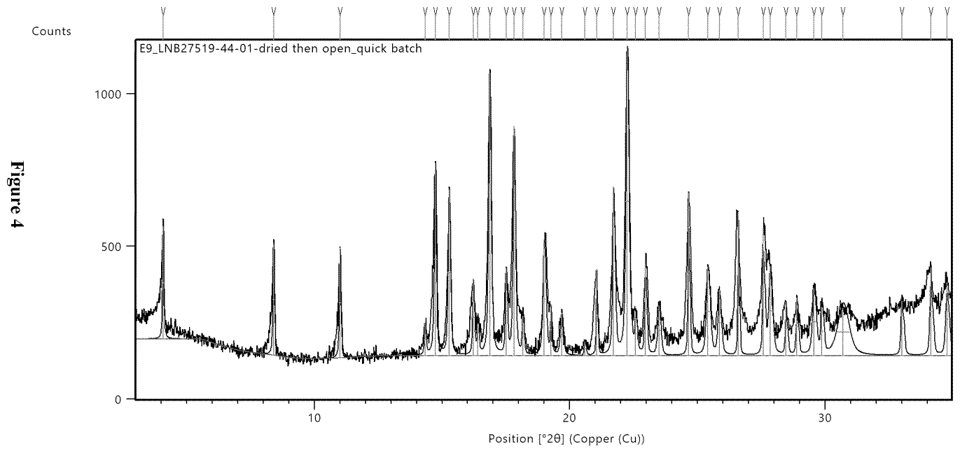

### DETAILED DESCRIPTION OF THE INVENTION

We have now found new processes for the preparation of crystalline forms of ivabradine HCl and in particular of the anhydrous delta-d crystalline form, which involve the removal of the crystallization solvent under controlled temperature and humidity conditions or, alternatively, by treatment with supercritical CO₂.

We therefore found a process for preparing the delta-d form of ivabradine HCl, which includes:
a) crystallization of crude ivabradine HCl in a suitable solvent to yield the corresponding crystalline solvate;
b) the removal of the crystallization solvent by exposure of the crystalline solvate, optionally subjected to a preventive drying, in an inert atmosphere with controlled relative humidity optionally followed by drying; or alternatively
b′) the removal of the crystallization solvent by exposure to a supercritical CO₂ flow.

Raw ivabradine HCl used in step a) can be obtained according to known processes and in particular according to the processes described in EP 0 534 859 or it can be obtained by salification of ivabradine free base with HCl gas in a suitable solvent.

Crude ivabradine HCl can be obtained by treatment of ivabradine HCl with acetonitrile to give an acetonitrile solvate, or by salification of ivabradine free base with gaseous HCl in the presence of C1-C5 alcohols, such as ethanol, isopropanol, 2-methyl-2-butanol methyl ethyl ketone, acetonitrile or mixtures thereof.

In step a), crude ivabradine HCl is suspended and then dissolved by heating in a suitable solvent equal to or different from the step of preparation of the crude, and preferably selected from acetonitrile, C1-C5 alcohols, methylethylketone or mixtures thereof. The solution thus obtained is left to cool until the solid compound precipitates, which is isolated and possibly subjected to mild drying. Said solid compound is a solvate of ivabradine HCl with the solvent used.

When the solvent used in step a) is selected from acetonitrile, C1-C5 alcohols, preferably ethanol or isopropanol or 2-methyl-2-butanol, methyl ethyl ketone, the delta forms of solvates of acetonitrile, C1-C5 alcohol are respectively obtained, preferably ethanol solvate, 2-methyl-2-butanol solvate and isopropanol solvate, methyl ethyl ketone solvate optionally mixed with a certain quantity of anhydrous delta-d forms of ivabradine HCl. Said solvates of delta forms of ivabradine HCl optionally in mixture with the delta-d forms of ivabradine HCl can be exposed to an inert atmosphere with controlled relative humidity, leading to the formation of the delta-hydrated form which, after drying under vacuum, is transformed into the anhydrous form delta-d of ivabradine HCl characterized by a content of methyl ethyl ketone, ethanol, 2-methyl-2-butanol or isopropanol lower than 5000 ppm, preferably lower than 2000 ppm; acetonitrile lower than 400 ppm preferably lower than 100 ppm, and relative humidity (Karl Fischer) KF<0.5.

Alternatively, said delta forms of ivabradine HCl solvates, optionally in admixture with delta-d forms of ivabradine HCl, preferably delta forms of ivabradine HCl solvate of acetonitrile, or C1-C5 alcohols solvates, preferably ethanol, 2-methyl-2-butanol o isopropanol, or methyl ethyl ketone solvates, optionally in admixture with delta-d forms of ivabradine HCl, can be subjected to a supercritical CO₂ flow which leads to the removal of the solvent and the transition to the anhydrous delta-d form of ivabradine HCl.

It has therefore been shown that if delta forms of ivabradine HCl solvates optionally in admixture with delta-d forms of ivabradine HCl, preferably delta forms of ivabradine HCl acetonitrile solvate, or C1-C5 alcohols solvates, preferably ethanol, 2-methyl-2-butanol or isopropanol, or methyl ethyl ketone solvates, optionally in admixture with delta-d forms of ivabradine HCl, are subjected to a supercritical CO₂ flow under suitable conditions of pressure, flow and time, these are transformed into the anhydrous delta-d form having a content of methyl ethyl ketone, ethanol, 2-methyl-2-butanol or isopropanol lower than 5000 ppm, preferably lower than 2000 ppm; content of acetonitrile lower than 400 ppm preferably lower than 100 ppm even more preferably lower than 40 ppm, and KF<0.5.

Therefore, an object of the invention is a process for the preparation of delta-d crystalline forms of ivabradine HCl, which includes:
a) crystallization of raw ivabradine HCl in a solvent selected from acetonitrile, methyl ethyl ketone, C1-C5 alcohols, to give the corresponding crystalline solvate;
b) the removal of the crystallization solvent by exposure of the crystalline solvate obtained in step a), optionally subjected to a preventive drying, in an inert atmosphere with controlled relative humidity, followed by drying; or alternatively
b′) the removal of the crystallization solvent by exposure to a supercritical CO₂ flow.

Preferably, the crystalline solvate of ivabradine HCl prepared in step a) comprises delta forms of acetonitrile solvate, C1-C5 alcohols solvates, methyl ethyl ketone solvate optionally in admixture with delta-d forms of ivabradine HCl. More preferably the crystalline solvate of ivabradine HCl prepared in step a) comprises mixtures of delta forms of acetonitrile solvate, ethanol solvate, 2-methyl-2-butanol solvates, isopropanol solvate optionally in admixture with delta-d forms of ivabradine HCl.

The optional preventive drying of the crystalline product obtained can be carried out under vacuum at a temperature between room temperature and 80° C., more preferably between 35° C. and 70° C., even more preferably between 40° C. and 60° C.

Preferably, step b) is carried out in an inert atmosphere with relative humidity between 15 and 65%, more preferably between 20 and 55%, even more preferably between 30 and 45%, for a time between 5 and 36 hours, preferably between 10 and 24 hours, at a temperature between 10 and 40° C., preferably between 20 and 30° C.

Preferably, step b) is carried out on mixtures of Ivabradine HCl delta forms of solvates of acetonitrile, ethanol, isopropanol, 2-methyl-2-butanol or of methyl ethyl ketone optionally in admixture with delta-d forms of ivabradine HCl.

Preferably, step b′) is carried out at a supercritical CO2 flow at a pressure between 70 bar and 140 bar, preferably between 85 bar and 120 bar; at a temperature between 40° C. and 100° C., preferably between 70° C. and 90° C., with a flow suitably chosen according to the equipment used.

For example, for a reactor having internal dimensions 7 mm×150 mm and filling≥70%, the flow is between 1 and 20 mL/min, preferably between 2 and 10 mL/min, even more preferably between 2 and 5 mL/min.

In step b′) ivabradine HCl is obtained in delta-d form with an acetonitrile content lower than 200 ppm, preferably lower than 100 ppm, more preferably lower than 40 ppm, even more preferably lower than 10 ppm; or a content of ethanol, 2-methyl-2-butanol or isopropanol lower than 5000 ppm, preferably lower than 2000 ppm and KF<0.5.

The process of the invention allows to obtain ivabradine HCl in delta-d form starting from a raw solvate of ivabradine HCl which can be crystallized from a solvent selected from C1-C5 alcohols, preferably selected from ethanol, 2-methyl-2-butanol and isopropanol; acetonitrile and methyl ethyl ketone to give the corresponding crystalline solvate which, subjected to an optional drying, and subsequent exposure to controlled relative humidity, provides a hydrated form which can be optionally dried to give the anhydrous form.

Surprisingly, the crude solvate of ivabradine HCl can be converted to a delta form of a solvate of ivabradine HCl derived from the crystallization of the crude ivabradine HCl with C1-C5 alcohols, preferably ethanol, 2-methyl-2-butanol or isopropanol, methyl ethyl ketone or acetonitrile and subsequently transformed into a hydrated form using controlled relative humidity, said hydrated form when suitably dried leads to the formation of the anhydrous delta-d polymorph of ivabradine HCl with purity requirements and residual solvent content well below the limits set by the ICH guidelines.

Alternatively, said crude crystalline solvate of ivabradine HCl can be subjected to drying and subsequently to a supercritical CO₂ flow which allows to remove the crystallization solvent and to provide the anhydrous delta-d form of ivabradine HCl with residual acetonitrile content up to less than 5 ppm, content of methyl ethyl ketone, ethanol, 2-methyl-2-butanol or isopropanol lower than 5000 ppm, preferably lower than 2000 ppm.

In order to better illustrate the present invention, the following examples are now provided.

### Analytical

X-ray diffractometric analysis—powder process (XRPD)

The samples, before being analyzed, were subjected to a gentle grinding in an agate mortar and then analyzed by X-ray diffractometry, with the following instrumental characteristics:
Philips diffractometer model PW1800/10
X′Pert High Score data processing software—v. 2.0a (PANalytical)
Radiation Cu Kα (Kα1=1.54060 Å Kα2=1.54439 Å)
graphite monochromator
diverging automatic slide
generator power: 45 Kv, 35 mA
scan interval: 2°-65° 2 θ
scan speed (step): 0.02° 2 θ/sec
counting time per step: 1.0 sec

The samples are analyzed in the scan interval: 2°-65° 2 θ.

### DSC Analysis (Differential Scanning Calorimetry)

DSC analyzes were conducted using METTLER TOLEDO's DSC 822e instrument. The experiments were conducted with a heating ramp of 10.0° C./min in the range 30-350° C. and with a nitrogen flow of 40 ml/min. 40 μL aluminum crucibles with perforated lid were used.

### IR Analysis

The IR spectra were recorded using a JASCO FT-IR 460 Plus spectrophotometer. The samples were prepared by grinding about 5 mg of sample with about 500 mg of KBr and analyzed in the range 4000-400 cm⁻¹ with a resolution of 4 cm⁻¹.

### Example 1: Treatment with Supercritical CO₂

Ivabradine HCl (delta solvated form of acetonitrile) is loaded into the extraction chamber which is then connected to the plant. The extraction phase is started at 80° C. by varying the parameters of temperature, CO₂ flow and time as shown in Table 1 below.

TABLE 1

| | Weight | Pressure | Flow | Temperature | Time | Acetonitrile |
|---|---|---|---|---|---|---|
| N. | (g) | (bar) | (mL/min) | (° C.) | (h) | (ppm) |
| Delta form of ivabradine HCl acetonitrile solvate | | | | | | 67118.5 |
| A | 1.391 | 85 | 5 | 80 | 6 | 134.9 |
| B | 1.364 | 120 | 2 | 80 | 6 | 40.5 |
| C | 1.373 | 85 | 2 | 80 | 12 | <1.5 |
| D | 1.317 | 120 | 5 | 80 | 12 | 3.6 |

### Example 2: Delta-d form of Ivabradine HCl

In a flask, 1 g of acetonitrile solvated of ivabradine HCl (2 mmol) is suspended in 6 mL of acetonitrile and 0.2 mL of water. The suspension is heated to reflux until it is completely dissolved. Subsequently the solution is cooled to 20-25° C. observing precipitation of the product. The obtained suspension is left under stirring for at least 1 hour. The desired product is then isolated by filtration and washed with 0.5 mL of acetonitrile (delta form).

The product obtained is kept for 20 hours at a relative humidity of 43% (in the presence of a saturated solution of potassium carbonate) and subsequently dried under vacuum at 40° C. for 15 hours, so as to obtain the desired delta-d form (yield 90,0%).

### Example 3: Delta-d form of Ivabradine HCl

In a flask, 1 g of ivabradine HCl ethanol solvate (2 mmol) is suspended in 5 mL of absolute ethanol. The suspension is heated to reflux until it is completely dissolved. Subsequently the solution is cooled to 20-25° C. observing precipitation of the product. The obtained suspension is left under stirring for at least 1 hour. The desired product is then isolated by filtration and washed with 0.5 mL of ethanol (delta form).

The product obtained is kept 8-10 hours at a relative humidity of 43% (in the presence of a saturated solution of potassium carbonate) and subsequently dried under vacuum at 40° C. for 15 hours, so as to obtain the desired delta-d form (yield 90.0%).

### Example 4: Delta-d form of Ivabradine HCl

In a flask, 1 g of ivabradine HCl isopropanol solvate (2 mmol) is suspended in 5 mL of isopropanol. The suspension is heated to reflux until it is completely dissolved. Subsequently the solution is cooled to 20-25° C. observing precipitation of the product. The obtained suspension is left under stirring for at least 1 hour. The desired product is then isolated by filtration and washed with 1 mL of isopropanol (delta form).

The product obtained is kept 8-10 hours at a relative humidity of 43% (in the presence of a saturated solution of potassium carbonate) and subsequently dried under vacuum at 40° C. for 15 hours, so as to obtain the desired delta-d form (yield 85.0%).

### Example 5: Delta-d form of Ivabradine HCl

In a flask, 1 g of Ivabradine HCl (2 mmol) is suspended in 5 mL of absolute ethanol. The suspension is heated to reflux until it is completely dissolved. Subsequently the solution is cooled to 20-25° C. observing precipitation of the product. The suspension is left under stirring for at least 1 hour. The desired product is then isolated by filtration after adding 3 mL of ethanol and washed with 0.5 mL of ethanol (delta form).

The product obtained is dried under vacuum at 55° C. for 15 hours, kept for 12 hours at a relative humidity of 43% (in the presence of a saturated solution of potassium carbonate) and subsequently kept for 15 hours under a flow of dry nitrogen, so as to obtain the desired delta-d form (yield 90.0%).

### Example 6: Delta-d form of Ivabradine HCl

In a flask 1 g of Ivabradine HCl (2 mmol) is suspended in 5 mL of absolute ethanol. The suspension is heated to reflux until it is completely dissolved. Subsequently the solution is cooled to 20-25° C. observing precipitation of the product. The suspension is left under stirring for at least 1 hour. The desired product is then isolated by filtration after adding 3 mL of ethanol and washed with 0.5 mL of ethanol (delta form).

The obtained product is kept 15 hours under nitrogen flow at a relative humidity of 43% (in the presence of a saturated solution of potassium carbonate) and subsequently dried under vacuum at 40° C. for 15 hours, so as to obtain the delta-d form desired (yield 90.0%).

1. A process for the preparation of delta-d crystalline forms of ivabradine HCl, characterized from a powder X-ray diffractogram showing peaks at values of the diffraction angles 2 θ of 14.6, 15.3, 17.2, 18.1 and 21.4, which comprises:
a) crystallization of crude ivabradine HCl in a solvent selected from acetonitrile, methyl ethyl ketone, C1-C5 alcohols, to give the respective crystalline solvate;
b) removal of the crystallization solvent by exposure of the crystalline solvate obtained in a), optionally subjected to a first drying, to an inert atmosphere with controlled relative humidity, followed by, drying; or alternatively
b′) removal of the crystallization solvent by exposure to a supercritical CO₂ flow.

2. The process according to claim 1, wherein the C1-C5 alcohols are ethanol, isopropanol or 2-methyl-2-butanol.

3. The process according to claim 1 wherein the solvent is selected from ethanol and isopropanol.

4. The process according to claim 1, wherein the crystalline solvate obtained in a) is subjected to a first drying under vacuum at a temperature between room temperature and 80° C.

5. The process according to claim 1, wherein the removal of the crystallization solvent is carried out under inert atmosphere with relative humidity comprised between 15 and 65%, for a time between 5 and 36 hours, at a temperature between 10° C. and 40° C.

6. The process according to claim 1, wherein the crystalline solvate of ivabradine HCl prepared in a) comprises delta forms of solvates of acetonitrile, C1-C5 alcohols, or methyl ethyl ketone optionally in admixture with delta-d forms of ivabradine HCl.

7. The process according to claim 1 wherein step b) is carried out on mixtures of delta forms of solvates of acetonitrile, ethanol, 2-methyl-2-butanol, isopropanol, methyl ethyl ketone optionally in mixture with delta-d forms of ivabradine HCl.

8. The process according to claim 1, wherein step b) the delta-hydrated form of ivabradine HCl having KF between 5% and 10% is obtained.

9. The process according to claim 1, wherein the removal of the crystallization solvent is carried out by treatment with supercritical CO₂ at a pressure between 70 bar and 140 bar at a temperature between 40° C. and 1.00° C.

10. The process according to claim 1, wherein the delta-d form of ivabradine HCl is obtained having an acetonitrile content lower than 200 ppm or a content of ethanol or isopropanol or 2-methyl-2-butanol lower than 5000 ppm, and KF<0.5.

11. The process according to claims 2, wherein the crystalline solvate obtained in a) is subjected to a first drying under vacuum at a temperature between room temperature and 80° C.

12. The process according to claims 3, wherein the crystalline solvate obtained in a) is subjected to a first drying under vacuum at a temperature between room temperature and 80° C.

13. The process according to claim 2, wherein the removal of the crystallization solvent is carried out under inert atmosphere with relative humidity comprised between 15 and 65%, for a time between 5 and 36 hours, at a temperature between 10° C. and 40° C.

14. The process according to claim 3, wherein the removal of the crystallization solvent is carried out under inert atmosphere with relative humidity comprised between 15 and 65%, for a time between 5 and 36 hours, at a temperature between 10° C. and 40° C.

15. The process according to claim 4, wherein the removal of the crystallization solvent is carried out under inert atmosphere with relative humidity comprised between 15 and 65%, for a time between 5 and 36 hours, at a temperature between 10° C. and 40° C.

16. The process according to claim 2, wherein the crystalline solvate of ivabradine HCl prepared in a) comprises delta forms of solvates of acetonitrile, C1-C5 alcohols, or methyl ethyl ketone optionally in admixture with delta-d forms of ivabradine HCl.

17. The process according to claim 3, wherein the crystalline solvate of ivabradine HCl prepared in a) comprises delta forms of solvates of acetonitrile, C1-C5 alcohols, or methyl ethyl ketone optionally in admixture with delta-d forms of ivabradine HCl.

18. The process according to claim 4, wherein the crystalline solvate of ivabradine HCl prepared in a) comprises delta forms of solvates of acetonitrile, C1-C5 alcohols, or methyl ethyl ketone optionally in admixture with delta-d forms of ivabradine HCl.

19. The process according to claim 5, wherein the crystalline solvate of ivabradine HCl prepared in a) comprises delta forms of solvates of acetonitrile, C1-C5 alcohols, or methyl ethyl ketone optionally in admixture with delta-d forms of ivabradine HCl.

20. The process according to claim 2, wherein step b) is carried out on mixtures of delta forms of solvates of acetonitrile, ethanol, 2-methyl-2-butanol, isopropanol, methyl ethyl ketone optionally in mixture with delta-d forms of ivabradine HCl.
